(19) United States
(12) Patent Application Publication
(10) Pub. No.: US 2023/0193343 A1
(43) Pub. Date: Jun. 22, 2023

(54) APPARATUS AND METHOD FOR MEASURING SALIVARY GLUCOSE LEVELS

(71) Applicant: AXBIO INC., SANTA CLARA, CA (US)

(72) Inventors: PRIYANKA AGRAWAL, SANTA CLARA, CA (US); VLADIMIR BASHKIROV, SANTA CLARA, CA (US); STEPHEN F. DREYER, SANTA CLARA, CA (US); YUN HE, SANTA CLARA, CA (US); IGOR IVANOV, SANTA CLARA, CA (US); CHING JINN LIN, SANTA CLARA, CA (US); LANXIN SHEN, SANTA CLARA, CA (US); HUI TIAN, SANTA CLARA, CA (US)

(21) Appl. No.: 18/068,801

(22) Filed: Dec. 20, 2022

Related U.S. Application Data
(60) Provisional application No. 63/292,851, filed on Dec. 22, 2021.

(51) Int. Cl. C12Q 1/00; B01L 3/00

(52) U.S. Cl. CPC C12Q 1/006; B01L 3/502715; B01L 2300/0825; B01L 2300/0645; B01L 2300/027; B01L 2200/04

(57) ABSTRACT

Apparatus and methods for determining the blood glucose concentration of a patient are disclosed A saliva glucose measurement strip is used in conjunction with a glucose measurement device to measure an electrochemical signal of a saliva sample and the measured electrochemical signal is correlated to a blood glucose convention.

This application claims the benefit under 35 U.S.C. § 119(e) of U.S. Provisional Application No. 63/292,851 filed on Dec. 22, 2021, which is incorporated by reference in its entirety.

### FIELD

The present disclosure relates to apparatus and methods for determining the blood glucose concentration of a patient. A saliva glucose measurement strip is used in conjunction with a glucose measurement device to measure an electrochemical signal of a saliva sample from a patient and the measured electrochemical signal is correlated to a blood glucose concentration.

### BACKGROUND

Self-monitoring of blood glucose is widely used to manage diseases such as diabetes. Simple to use, accurate, and non-invasive methods for determining blood glucose concentration can facilitate the ability of diabetic individuals to self-manage the disease.

### SUMMARY

According to the present invention, a saliva glucose measurement strip comprises a substrate comprising a first substrate portion, a second substrate portion, and a third substrate portion; a first printed circuit, wherein a first portion of the first printed circuit overlies the first substrate portion, a second portion of the first printed circuit overlies the second substrate portion, and a third portion of the first printed circuit overlies the third substrate portion; an insulator overlying the second portion of the first printed circuit; a second printed circuit overlying and electrically connected to the third portion of the first printed circuit; a filter overlying the second printed circuit; two inserts overlying the second printed circuit and coplanar with the filter; and a cover overlying the two inserts, a portion of the filter, and a portion of the insulator.

According to the present invention, glucose measurement devices comprise a housing; a processor; a display interconnected to the processor; an actuator interconnected to the processor; and a saliva glucose measurement strip insertion port wherein the insertion port is configured to interconnect the saliva glucose measurement strip according to the present invention.

According to the present invention, methods of determining a blood glucose concentration of a patient comprise: measuring an electrochemical signal of a saliva sample of a patient using the saliva glucose measurement strip provided by the present disclosure; and correlating the measured electrochemical signal to a blood glucose concentration to thereby determine the blood glucose concentration of the patient.

### BRIEF DESCRIPTION OF THE DRAWINGS

Those skilled in the art will understand that the drawings described herein are for illustration purposes only. The drawings are not intended to limit the scope of the present disclosure.

FIG. 1A shows an assembly view of an example of a saliva glucose measurement strip provided by the present disclosure.

FIG. 1B shows a top view of an example of a saliva glucose measurement strip provided by the present disclosure.

FIG. 2 shows a top internal view of a saliva glucose measurement strip provided by the present disclosure.

FIG. 3 is a schematic diagram of a saliva glucose measurement device provided by the present disclosure.

FIG. 4 is a flow diagram illustrating a method of using a saliva glucose measurement strip and measurement device provided by the present disclosure for determining a blood glucose concentration.

### DETAILED DESCRIPTION

For purposes of the following detailed description, it is to be understood that embodiments provided by the present disclosure may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

“Patient” refers to a human such as a human patient having diabetes or other disease or condition for which glucose monitoring can be useful.

A saliva glucose measurement device provided by the present is used in conjunction with a saliva glucose measurement strip to determine a patient's blood glucose concentration based on an electrochemical measurement of a saliva sample from the patient.

A saliva glucose measurement strip is used to measure am electrochemical signal of a saliva sample from a patient. A saliva glucose measurement strip includes electrodes configured to measure an electrochemical signal proportional to the glucose concentration in a saliva sample such as a sample of saliva in contact with electrodes.

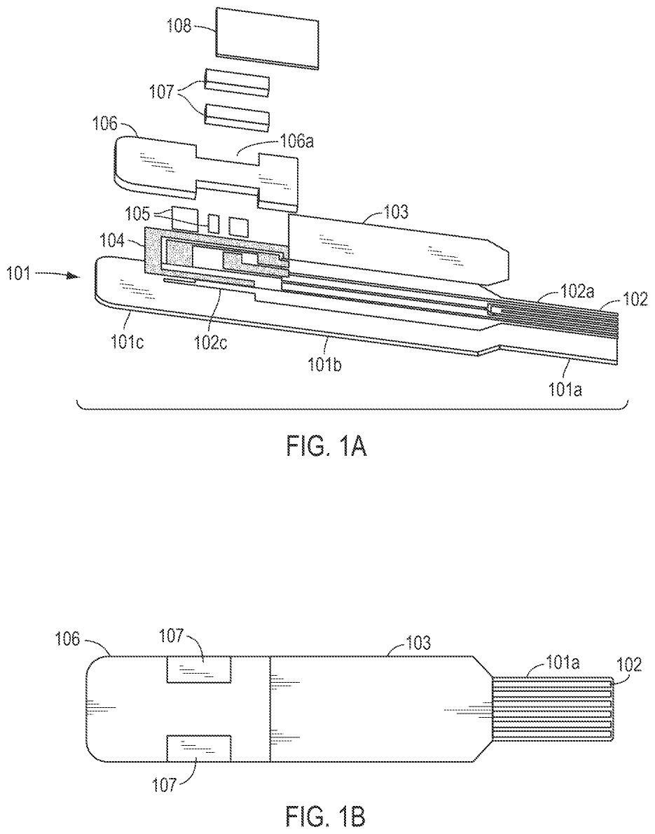

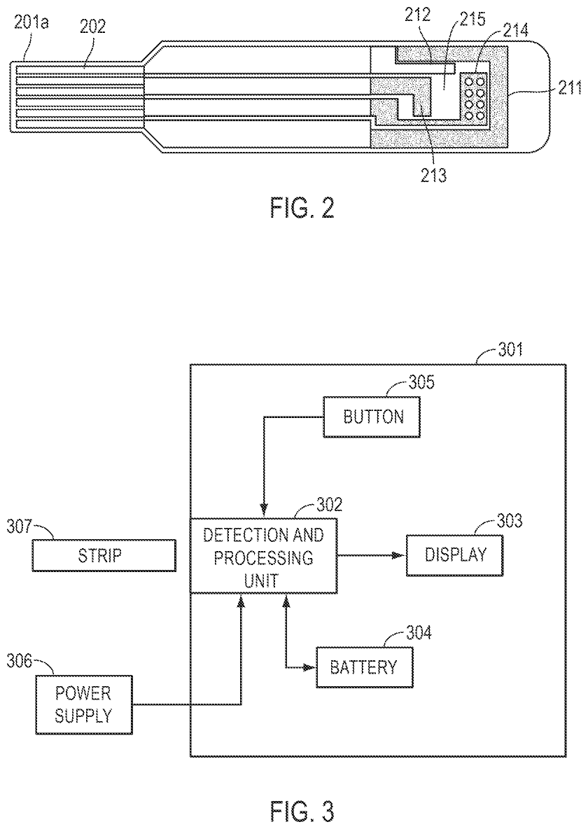

An example of a saliva glucose measurement strip provided by the present disclosure is shown in FIGS. 1A, 1B, and 2.

As shown in FIG. 1A, a saliva glucose measurement strip provided by the present disclosure can comprise a substrate 101 having a first substrate portion 101a, a second substrate portion 101b, and a third substrate portion 101c. A substrate can comprise any suitable electrically insulating material such as a polymeric material. A suitable polymeric material can be a thermoplastic or a thermoset. A substrate can have a length, for example, from 2 inches to 4 inches (50.8 mm to 101.6 mm), and a width, for example, from 0.25 inches to 1 inch (6.35 mm to 25.4 mm). A substrate can have a thickness, for example, less than 0.125 inches (3.17 mm).

A first printed circuit 102 can overlie the substrate. The first printed circuit can include an electrical interconnect such as interconnection fingers 102a overlying the first substrate portion 101a. Electrical interconnect 102a can be configured to electrically interconnect to a connector of a glucose measurement device. The first printed circuit 102 can be configured to independently interconnect to electrodes of a second printed circuit. The first printed circuit can be made from any suitable electrically conductive material. For example, the first printed circuit can be made from an electrically conductive ink. A suitable electrically conductive ink can comprise, for example, a silver ink, such as a silver ink comprising silver and silver chloride.

An insulator 103 can overlie a second portion of the first printed circuit overlying the second substrate portion 101b. The insulator 103 can physically protect the second portion of the first printed circuit. The insulator 103 can be made from any suitable insulating material such as a polymeric material, including thermoplastics and thermosets.

A second printed circuit 104 can overlie the third portion 102c of the first printed circuit 102. The second printed circuit 104 can include electrodes and each of the electrodes can be interconnected to a corresponding trace of the first printed circuit 102. The second printed circuit 104 can be made from any suitable electrically conductive material. For example, the second printed circuit can be made from an electrically conductive ink. Examples, of suitable electrically conductive inks for the second printed circuit include graphene conductive inks. For example, a suitable graphene conductive ink can be made from a composition comprising 1 mg/mL graphene and 0.2 mg/mL poly(3,4-ethylenedioxythiophene) polystyrene sulfonate (PRDOT:PSS) in N,N-dimethylformamide.

A second printed circuit 104 can include a reference electrode, two counter electrodes, and at least one working electrode. For example, a second printed circuit can include a reference electrode, two counter electrodes, and one working electrode; or a second printed circuit can include a reference electrode, two counter electrodes, and two working electrodes.

As shown in FIG. 1A, an enzyme coating 105 can overlie each of the working electrodes.

An enzyme coating 105 can comprise a ferrocyanide such as a Fe′ ferrocyanide.

An enzyme coating 105 can comprise a glucose oxidase. A glucose oxidase enzyme is an oxidase that catalyzes the oxidation of glucose to hydrogen peroxide and D-glucono-6-lactone.

An enzyme coating 105 can comprise a peroxidase. A peroxidase catalyzes the reaction of hydrogen peroxide to oxygen and water.

An enzyme coating 105 can comprise a ferrocyanide, a glucose oxidase and a peroxidase.

As an example, the electrochemical enzyme method can be based on the reaction of glucose with a glucose oxidase and potassium ferricyanide.

The glucose in the saliva can be catalytically oxidized to gluconic acid by a glucose oxidase on the enzyme coating, and glucose oxidase (GO) can then be converted to its reduced state at the same time. For example, the following reaction can take place at the coating: glucose+GO_{(OX)}→gluconic acid+GO_{(RED)}; where OX and RED represent the oxidation state and the reduced state, respectively.

The reduced glucose oxidase reduces the ferricyanide ions of the potassium ferricyanide to ferrocyanide ions. For example, the reaction can which can take place according to the following process: GO_{(RED)}+2M_{(OX)}→GO_{(OX)}+2M_{(RED)}+2H⁺; where M_{(OX)} and M_{(RED)} represent the ferricyanide ion and ferrocyanide ion, respectively.

When a certain voltage is applied to the electrodes, the ferrocyanide ions are reduced to ferricyanide ions and a current is generated. The reaction can be summarized as follows: 2M_{(RED)}→2M_{·(OX)}+2e⁻; where e⁻ represents electrons. The greater the number of free electrons generated, the greater the current, which is proportional to the glucose concentration.

An enzyme coating can be prepared from an enzyme composition.

For example, the enzyme coating can be prepared from an enzyme composition comprising 50 mM potassium ferrocyanide (Fe²⁺), 50 mM phosphate buffer, 1.6 units of UL niger glucose oxidase, and 2.5 unit of UL horseradish peroxidase at pH 7.0. The enzyme composition can be applied to the electrodes and dried to form an enzyme coating having a dried film thickness, for example, less than 50 μm, less than 25 μm, or less than 10 μm.

A suitable enzyme composition can comprise, for example from 0.6 units to 2.6 units of niger glucose oxidase, from 0.8 units to 2.4 units, from 1.0 units to 2.2 units, from 1.2 units to 2.0 units, or from 1.4 units to 1.8 units niger glucose oxidase.

A suitable enzyme composition can comprise, for example, from 1.5 units to 3.5 units horseradish peroxidase, from 1.7 units to 3.3 units, from 1.9 units to 3.1 units, from 2.1 units to 2.9 units, or from 2.3 units to 2.7 units horseradish peroxidase.

A suitable enzyme composition can comprise, for example from 0.6 units to 2.6 units niger glucose oxidase, from 0.8 units to 2.4 units, from 1.0 units to 2.2 units, from 1.2 units to 2.0 units, or from 1.4 units to 1.8 units niger glucose oxidase, and from 1.5 units to 3.5 units horseradish peroxidase, from 1.7 units to 3.3 units, from 1.9 units to 3.1 units, from 2.1 units to 2.9 units, or from 2.3 units to 2.7 units horseradish peroxidase.

A filter can overlie the third portion of the substrate, the third portion of the first printed circuit, and the second printed circuit. The filter can be configured to absorb saliva and transport or pass small molecules such as glucose through the filter to the electrodes.

As shown in FIG. 1A a filter overlies the second printed circuit. The filter 106 can be configured to retain a sample of saliva and direct small molecules within the saliva sample to a measurement well defined by the second printed circuit.

A filter can be selected to filter out large molecules such as proteins and to allow small molecules such as glucose to be transported or pass though the filter. A small molecule can have a molecular weight, for example, less than 500 Daltons, less than 400 Daltons, or less than 300 Daltons. A small molecule can be glucose. A filter can comprise a cellulose-based fiber, a hybrid material such as a polymeric fiber web with a hydrophilic foam matrix. Examples of suitable fiber materials include SureWick® C083 cellulose absorbent materials available from EMD Millipore®, and hybrid materials including filter materials available from Porex® Corporation.

A fitter can have a thickness, for example, from 0.25 mm to 2 mm, such as from 0.5 mm to 1. 5 mm.

The filter can be attached to the substrate using, for example, a clamp, an ultrasonic weld, a clip, or an adhesive.

A filter can include two cutouts 106a on either side of the filter such that when assembled the cutouts are situated over the reference electrode. The two cutouts are configured to accommodate two inserts.

Inserts 107 are fit into the cutouts in the filter 106. The inserts are made from a material that does not absorb and transfer saliva. For example, the inserts can be made from a thermoplastic or thermoset.

An insert is disponed in each of the two cutouts in the filter and overlie the reference electrode and the circuit interconnected to the reference electrode.

An insert can be formed from any suitable electrically insulating material that is not permeable to saliva. For example, an insert can be made of a thermoplastic or a thermoset

A cover 108 can overly the inserts, a portion of the filter 106 and a portion of the insulator 103. The cover can physically protect the region of the measurement strip in the vicinity of the electrodes and can ensure that saliva contacting the electrodes of the strip is transported from the distal end of the filter though the filter and into the measurement region.

A cover can be made of any suitable electorally insulating material such aa s a thermoplastic or a thermoset.

A top view of an assembled saliva glucose measurement strip is shown in FIG. 1B. FIG. 1B includes interconnection fingers 102 overlying the first portion of the first printed circuit 101a, insulator 103 in the second portion of the measurement strip, and a filter 106 toward the third portion of the measurement glucose strip. Two inserts 107 are disposed in cutouts on either side of the filter 106.

An internal view of a saliva glucose measurement strip provided by the present disclosure is shown in FIG. 2. The view shows the circuitry underlying the insulator and the filter.

The view shown in FIG. 2 includes a first portion of substrate 201a with overlying interconnection figures of first printed circuit 202. The interconnection fingers are independently interconnected to a counter electrode 211, a reference electrode 212, a first working electrode 213, and a second working electrode 214. The electrodes define a measurement region 215.

An enzyme coating is disposed on the working electrodes.

An enzyme coating is not disposed on the reference electrode and the two counter electrodes.

A glucose measurement strip provided by the present disclosure can comprise, for example, at least one working electrode such as one or two working electrodes, two counter electrodes, and a reference electrode.

A glucose measurement strip is used in conjunction with a glucose measurement device to determine a patient's blood glucose concentration.

A schematic diagram of an example of a saliva glucose measurement device provided by the present disclosure is shown in FIG. 3. The measurement device includes a housing 301, a detection/processing unit 302, a display 303 interconnected to the detection/processing unit 302, a power source 304 such as a battery interconnected to the detection/processing unit 302, an actuator 305 such as a button, an optional external power source 306 interconnected to the detection/processing unit 302, and an insertion port (not shown) configured to accept a saliva glucose measurement strip 307 provided by the present disclosure.

A measurement device can further include a memory unit either integrated with the detection/processing unit or as a separate memory unit.

The detection/processing unit 302 and internal power sources 304 can be retained within the housing 301, and the actuator 305 and display 303 can be mounted on the housing.

The insertion port can include a measurement strip connector configured to interconnect the interconnect fingers of the glucose measurement strip with the detection/processing unit and a power source.

Methods provided by the present disclosure comprise using a saliva glucose measurement strip and a glucose measurement device to determine a blood glucose concentration of a patient. The blood glucose concentration is derived from an electrochemical signal measured using the saliva glucose measurement strip that is proportional to the amount of glucose in a patient's saliva.

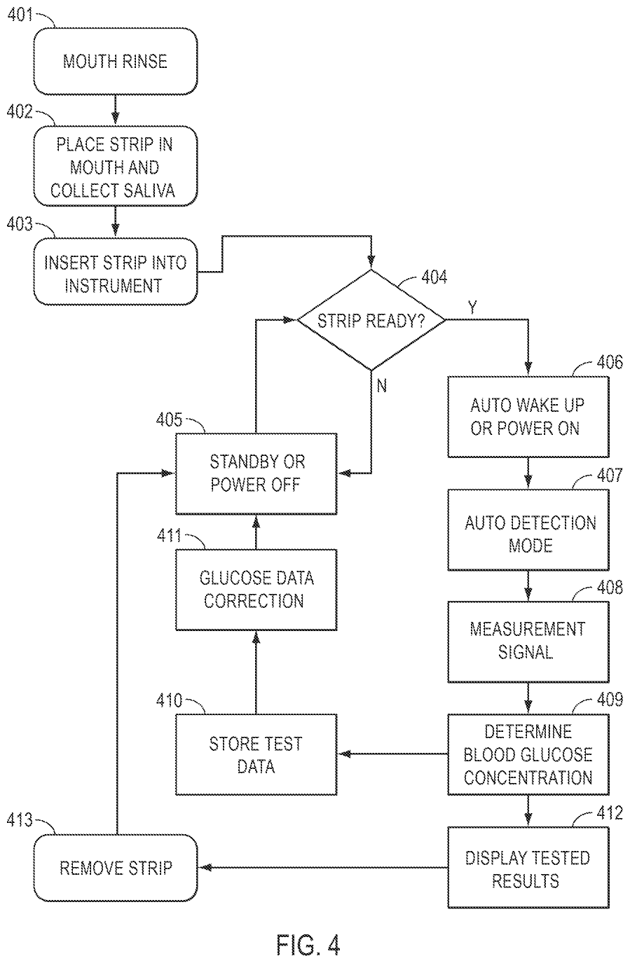

A flow diagram of an example of a method for determining a blood glucose concentration is shown in FIG. 4.

First, a saliva sample can be applied on the filter of the measurement strip. The saliva sample can be applied to the filter by inserting the third portion of the measurement strip into a patient's mouth. Before inserting the measurement strip into a patient's mouth, the patient can rinse the mouth with a suitable liquid such as water. The third portion of the measurement strip can then be inserted, for example, under the tongue or adjacent the parotid gland to obtain a saliva sample. The patient or a person other than the patient can insert the glucose measurement strip in the patient's mouth to obtain a saliva sample. After the distal portion of the filter is coated with saliva, the measurement strip can be removed from the patient's mouth. Alternatively, a saliva sample in a container can be removed and coated onto the distal portion of a filter using any suitable device such as a swab or a pipette.

After the third portion of the filter is covered with a saliva sample, the first portion of the measurement strip can be inserted into the insertion port of the glucose measurement device to cause the interconnection fingers of the measurement strip to engage with a connector. The connecter interconnects the saliva glucose measurement strip to the detection/processing unit of the glucose measurement device.

After insertion, the measurement device can be activated, either automatically or manually, to power the electrodes of the measurement strip.

Compounds contained within the saliva sample such as glucose can be transported through the filter to the measurement region and to contact the electrodes. An electrochemical signal is generated by the electrodes that is proportional to the glucose concentration of the saliva sample.

The measured saliva electrochemical signal is input into a blood glucose correlation algorithm to determine the blood glucose value corresponding to the measured saliva glucose value. The electrochemical signal is generated using chronoamperometry.

As shown in FIG. 4, a method of determining a blood glucose concentration can include the steps of a patient rinsing the mouth 401, placing a glucose measurement strip in the rinsed mouth and applying saliva to the measurement strip 402, removing the measurement strip from the mouth (not shown) and inserting the measurement strip into the insertion port of the glucose measurement device 403, detecting, by the measurement device, whether the measurement strip is interconnected 405; and if the measurement strip is not connected, maintaining the measurement device in the standby or power-off mode, or transition to the power off mode 405; or if the measurement strip is connected, automatically transitioning to the power-on mode 406. In the power-on mode 406, the measurement mode is activated 407, and an electrochemical signal is generated by the electrodes 408. The electrochemical signal is then input to a blood glucose correlation algorithm to determine the blood glucose concentration based on the electrochemical signal generated by the saliva sample 409. The determined blood glucose concentration is then presented on the display 412.

The calculated blood glucose concentration can be stored 410 and if necessary the glucose correlation algorithm can be corrected and the corrected algorithm stored.

After the blood glucose concentration is determined and displayed, the measurement strip can be removed from the measurement device 413, and the measurement device returned to the standby or power-off mode 405.

The electrochemical parameters of the saliva sample can be determined using chronoamperometry.

The electrodes of the measurement strip are configured to generate an electrochemical signal that is proportional to the glucose concentration of the saliva sample in the measurement region. Because the constituents of saliva can vary from individual to individual, and the saliva composition can influence the electrochemical signal generated by the electrodes, methods provided by the present disclosure include generating an individualized saliva glucose to blood glucose correlation algorithm.

The glucose correlation algorithm can be generated using various methods.

In one method a glucose correlation algorithm can be generated by (1) measuring the electrochemical signal generated by various saliva samples, (2) simultaneously measuring the plasma glucose concentration directly, and (3) deriving an algorithm correlating the measured saliva electrochemical signal to the measured blood glucose concentration.

In another method, a set of artificial saliva samples having known glucose concentrations can be prepared. The electrotechnical signal generated by each of the artificial saliva samples can be used derive a calibration algorithm correlating the measured electrochemical signal to the saliva glucose concentration for a measurement strip.

In another calibration method, a set of buffered solutions having known concentrations of glucose can be prepared and applied to the filter of a measurement strip. An algorithm can be derived correlating the measured electrochemical signal to the known glucose concentration for the various samples. The derived calibration algorithm can then be used to establish the glucose concentration based on the measured electrochemical signal of a sample. This method can be used to derive a correlation algorithm for a few saliva glucose measurement trips from a common production lot and the derived correlation algorithm used to determine the blood glucose concentration for each of the blood glucose measurement strips in the common production lot.

In another method, a known amount of glucose (A) at concentration C_{A} is applied to a second working electrode (WE2) and the signal measured. Then, using the filter, a first working electrode (WE1) generates a signal X=a×C of an unknown concentration of glucose. The sample liquid moves from WE1 to WE2 with a time delay, and WE2 generates a single Y=a×(C+C_{A}). From the known volumes of X, Y, and C_{A}, the absolute value for C can be determined.

Two correlations can be involved in determining a blood glucose concentration of a patient based on an electrochemical signal generated by a measurement strip provided by the present disclosure.

First, the correlation between a measured electrochemical signal and a glucose concentration in a saliva sample can be established; and second a correlation between the saliva glucose connotation and the blood glucose concentration can be established.

After the saliva/blood glucose correlation algorithm is derived and stored, the blood glucose concentration can be determined based on the electrochemical signals measured using the saliva glucose measurement strip.

The glucose correlation algorithm can be stored, for example, as an algorithm, or as a correlation table.

### Aspects

The invention is further defined by one or more of the following aspects.

Aspect 1. A saliva glucose measurement strip, wherein the measurement strip comprises: a substrate comprising a first substrate portion, a second substrate portion, and a third substrate portion; a first printed circuit, wherein a first portion of the first printed circuit overlies the first substrate portion, a second portion of the first printed circuit overlies the second substrate portion, and a third portion of the first printed circuit overlies the third substrate portion; an insulator overlying the second portion of the first printed circuit; a second printed circuit overlying and electrically connected to the third portion of the first printed circuit; a filter overlying the second printed circuit; two inserts overlying the second printed circuit and coplanar with the filter; and a cover overlying the two inserts, a portion of the filter, and a portion of the insulator.

Aspect 2. The measurement strip of aspect 1, wherein the substrate comprises a polymeric material.

Aspect 3. The measurement strip of any one of aspects 1 to 2, wherein the first printed circuit comprises an electrically conductive ink.

Aspect 4. The measurement strip of any one of aspects 1 to 3, wherein a first portion of the first printed circuit comprises electrical interconnects.

Aspect 5. The measurement strip of any one of aspects 1 to 4, wherein the insulator comprises a polymeric material.

Aspect 6. The measurement strip of any one of aspects 1 to 5, wherein the second printed circuit comprises an electrically conductive ink.

Aspect 7. The measurement strip of any one of aspects 1 to 5, wherein the second printed circuit comprises a graphene ink.

Aspect 8. The measurement strip of aspect 7, wherein the graphene ink comprises graphene and poly(3,4-ethylenedioxythiophene) polystyrene sulfonate (PRDOT:PSS).

Aspect 9. The measurement strip of any one of aspects 1 to 8, wherein the second printed circuit comprises a reference electrode, two counter electrodes, and at least one working electrode.

Aspect 10. The measurement strip of aspect 9, wherein the second printed circuit comprises a first working electrode and a second working electrode.

Aspect 11. The measurement strip of any one of aspects 9 and 10, comprising an electrochemical enzyme coating overlying each of the working electrodes.

Aspect 12. The measurement strip of aspect 11, wherein the electrochemical enzyme coating comprises potassium ferrocyanide, niger glucose oxidase, and horseradish peroxidase.

Aspect 13. The measurement strip of any one of aspects 1 to 12, wherein the filter comprises a material configured to filter out large molecules and to transport small molecules.

Aspect 14. The measurement strip of any one of aspects 1 to 13, wherein each of the inserts comprises an electrically insulating polymeric material.

Aspect 15. The measurement strip of any one of aspects 1 to 14, wherein the cover comprises an electrically insulating polymeric material.

Aspect 16. A glucose measurement device, comprising: a housing; a processor; a display interconnected to the processor; an actuator interconnected to the processor; and a saliva glucose measurement strip insertion port, wherein the insertion port is configured to interconnect the saliva glucose measurement strip of any one of aspects 1 to 15 to the processor.

Aspect 17. A method of determining a blood glucose concentration of a patient, comprising: measuring an electrochemical signal of a saliva sample of a patient using the saliva glucose measurement strip of any one of aspects 1 to 15; and correlating the measured electrochemical signal to a blood glucose concentration to thereby determine the blood glucose concentration of the patient.

Aspect 18. The method of aspect 17, wherein, before measuring, applying the saliva sample to the filter of the saliva glucose measurement strip.

Aspect 19. The method of any one of aspects 17 to 18, wherein measuring comprises using a glucose measuring device, wherein the measuring device comprises: a housing; a processor; a display interconnected to the processor; an actuator interconnected to the processor; and a saliva glucose measurement strip insertion port, wherein the insertion port is configured to interconnect the saliva glucose measurement strip to the processor.

Aspect 20. The method of any one of aspects 17 to 19, wherein, before measuring, inserting the first portion of the saliva glucose measurement strip into the injection port to electrically interconnect the first and second printed circuits to the processor.

Aspect 21. The method of any one of aspects 17 to 20, wherein correlating comprises using correlation algorithm that correlates a measured electrochemical signal to the blood glucose concentration.

Aspect 22. The method of aspect 21, wherein the correlation algorithm is derived based on measured blood concentrations.

Aspect 23. The method of any one of aspects 17 to 22, wherein the correlation algorithm is derived based on model saliva samples having known glucose concentrations.

Aspect 24. The method of any one of aspects 17 to 23, wherein the correlation algorithm is derived based on compositions having known glucose concentrations.

Finally, it should be noted that there are alternative ways of implementing the embodiments disclosed herein. Accordingly, the present embodiments are to be considered as illustrative and not restrictive. Furthermore, the claims are not to be limited to the details given herein and are entitled to their full scope and equivalents thereof.

What is claimed is:

1. A saliva glucose measurement strip, wherein the measurement strip comprises:
a substrate comprising a first substrate portion, a second substrate portion, and a third substrate portion;
a first printed circuit, wherein a first portion of the first printed circuit overlies the first substrate portion, a second portion of the first printed circuit overlies the second substrate portion, and a third portion of the first printed circuit overlies the third substrate portion;
an insulator overlying the second portion of the first printed circuit;
a second printed circuit overlying and electrically connected to the third portion of the first printed circuit;
a filter overlying the second printed circuit;
two inserts overlying the second printed circuit and coplanar with the filter; and
a cover overlying the two inserts, a portion of the filter, and a portion of the insulator.

2. The measurement strip of claim 1, wherein the substrate comprises a polymeric material.

3. The measurement strip of claim 1, wherein the first printed circuit comprises an electrically conductive ink.

4. The measurement strip of claim 1, wherein a first portion of the first printed circuit comprises electrical interconnects.

5. The measurement strip of claim 1, wherein the insulator comprises a polymeric material.

6. The measurement strip of claim 1, wherein the second printed circuit comprises an electrically conductive ink.

7. The measurement strip of claim 1, wherein the second printed circuit comprises a graphene ink.

8. The measurement strip of claim 7, wherein the graphene ink comprises graphene and poly(3,4-ethylenedioxythiophene) polystyrene sulfonate (PRDOT:PSS).

9. The measurement strip of claim 1, wherein the second printed circuit comprises a reference electrode, two counter electrodes, and at least one working electrode.

10. The measurement strip of claim 9, wherein the second printed circuit comprises a first working electrode and a second working electrode.

11. The measurement strip of claim 9, comprising an electrochemical enzyme coating overlying each of the working electrodes.

12. The measurement strip of claim 11, wherein the electrochemical enzyme coating comprises potassium ferrocyanide, niger glucose oxidase, and horseradish peroxidase.

13. The measurement strip of claim 1, wherein the filter comprises a material configured to filter out large molecules and to transport small molecules.

14. The measurement strip of claim 1, wherein each of the inserts comprises an electrically insulating polymeric material.

15. The measurement strip of claim 1, wherein the cover comprises an electrically insulating polymeric material.

16. A glucose measurement device, comprising:
a housing;
a processor;
a display interconnected to the processor;
an actuator interconnected to the processor; and
a saliva glucose measurement strip insertion port,
wherein the insertion port is configured to interconnect the saliva glucose measurement strip of claim 1 to the processor.

17. A method of determining a blood glucose concentration of a patient, comprising:
measuring an electrochemical signal of a saliva sample of a patient using the saliva glucose measurement strip of claim 1; and
correlating the measured electrochemical signal to a blood glucose concentration to thereby determine the blood glucose concentration of the patient.

18. The method of claim 17, wherein, before measuring, applying the saliva sample to the filter of the saliva glucose measurement strip.

19. The method of claim 17, wherein measuring comprises using a glucose measuring device, wherein the measuring device comprises:
a housing;
a processor;
a display interconnected to the processor;
an actuator interconnected to the processor; and
a saliva glucose measurement strip insertion port,
wherein the insertion port is configured to interconnect the saliva glucose measurement strip to the processor.

20. The method of claim 17, wherein, before measuring, inserting the first portion of the saliva glucose measurement strip into the injection port to electrically interconnect the first and second printed circuits to the processor.
